(19) United States
(12) Patent Application Publication
(10) Pub. No.: US 2025/0248696 A1
(43) Pub. Date: Aug. 7, 2025

(54) DEVICES, SYSTEMS, AND METHODS FOR TISSUE COLLECTION

(71) Applicant: Boston Scientific Medical Device Limited, Galway (IE)

(72) Inventors: Charudatta Chandrakant ARADHYE, Solapur (IN); Arun ADHIKARATH BALAN, Aluva (IN); Nachiket GOLE, Pune (IN); Nidhi DHINGRA, Gurgaon (IN); Subodh MOREY, Ponda (IN); Shrikant Vasant RAUT, Pune (IN); Samriddhi JAIN, Delhi (IN)

(73) Assignee: Boston Scientific Medical Device Limited, Galway (IE)

(21) Appl. No.: 19/046,940

(22) Filed: Feb. 6, 2025

Related U.S. Application Data
(60) Provisional application No. 63/550,736, filed on Feb. 7, 2024.

(51) Int. Cl. A61B 10/02

(52) U.S. Cl. CPC A61B 10/0266; A61B 2010/0225

(57) ABSTRACT

Medical systems and related methods useful for the retrieval of multiple tissue samples are described. The system may include a sleeve defining a lumen and an aperture proximate a distal end of the sleeve, an inner guide slidable along the lumen of the sleeve and rotatable relative to the sleeve via a handle at a proximal end of the inner guide. A distal end of the inner guide may have a closed atraumatic tip. The inner guide may define at least two cavities each having a shape corresponding to the aperture of the sleeve. The inner guide may be rotatable relative to the sleeve to align one of the at least two cavities with the sleeve to retrieve a tissue sample by proximal or distal movement of the inner guide relative to the sleeve.

### CROSS-REFERENCE TO RELATED APPLICATIONS

This application claims the benefit of priority to U.S. Provisional Application No. 63/550,736, filed on Feb. 7, 2024, which is incorporated by reference herein in its entirety.

### TECHNICAL FIELD

Aspects of the present disclosure generally relate to medical devices, system, and procedures related thereto. In particular, some aspects relate to medical systems, devices, and methods useful for collecting tissue samples, such as biopsy samples, within the body of a patient.

### BACKGROUND

During medical procedures, such as endoscopic or ureteroscopic procedures, tissue samples may be acquired from a subject's body for analysis. For example, in a biopsy procedure, tissue samples may be collected from an organ or a wall of a lumen in the body. Instruments like forceps, needles, or brushes may be introduced through the working channel of a scope or directly in the body to obtain the tissue samples. These tissue samples may be used to determine the presence of a pathological disorder, for example. However, biopsy procedures can be lengthy and difficult when more than one sample of tissue is necessary to make an accurate assessment. Furthermore, it may be challenging to obtain tissue samples from deep within the body at locations that are difficult to access.

### SUMMARY

The present disclosure includes medical instruments that may facilitate the retrieval of multiple tissue samples, e.g., without the need to withdraw the instrument between the retrieval of each sample.

For example, the present disclosure includes a medical instrument that comprises a sleeve defining a lumen and an aperture proximate a distal end of the sleeve. The inner guide may be slidable along the lumen of the sleeve and rotatable relative to the sleeve via a handle at a proximal end of the inner guide. A distal end of the inner guide may have a closed atraumatic tip. The inner guide may define at least two cavities proximate the distal end of the inner guide and a divider between each cavity and an adjacent cavity. Each cavity of the inner guide may have a shape corresponding to the aperture of the sleeve. The inner guide may be rotatable relative to the sleeve to align one of the at least two cavities with the sleeve to retrieve a tissue sample by proximal or distal movement of the inner guide relative to the sleeve.

According to some aspects, a distal portion of the medical instrument including the inner guide and the sleeve may have a preformed curvature relative to a longitudinal axis of the medical instrument. The preformed curvature may form an angle ranging from about 30° to about 90° relative to the longitudinal axis. The inner guide may define two cavities disposed on opposite sides of the inner guide. Each cavity of the inner guide may include a proximal ledge and a distal ledge, and at least one of the proximal ledge or the distal ledge may have a sharp edge configured to cut tissue. At least a portion of the sleeve defining the aperture may have a sharp edge configured to cut tissue. The sharp edge(s) of the ledge(s) and/or aperture may be a serrated edge. The inner guide may comprise Nitinol, stainless steel, or a polymer. Additionally or alternatively, the sleeve may comprise a polymer. In some examples, the handle of the inner guide may include a knob. Further, for example, a proximal end of the sleeve may include a hub, and the handle of the inner guide may be proximal to the hub. According to some aspects, the handle and the hub may include markings, e.g., to indicate a rotational orientation of the inner guide relative to the sleeve. Further, for example, a portion of the inner guide proximate the handle may include at least one marking to indicate a longitudinal position of the inner guide relative to the sleeve. In some examples, an internal stop may be between the inner guide and the sleeve to limit rotation of the inner guide relative to the sleeve. In some aspects, the inner guide may define three cavities proximate the distal end of the inner guide. For example, the three cavities may be disposed at 120° relative to one another.

The present disclosure also includes a medical instrument comprising a sleeve defining a lumen and an aperture proximate a distal end of the sleeve. The inner guide may be slidable along the lumen of the sleeve and rotatable relative to the sleeve via a handle at a proximal end of the inner guide. The inner guide may define at least two cavities proximate the distal end of the inner guide, each cavity of the at least two cavities being separated from an adjacent cavity and including a sharp edge configured to cut tissue. The cavity of the inner guide may have a shape corresponding to a shape of the aperture of the sleeve, the inner guide may be rotatable relative to the sleeve to align one of the at least two cavities with the sleeve to retrieve a tissue sample by proximal or distal movement of the inner guide relative to the sleeve.

According to some aspects, the proximal end of the sleeve includes a hub. The handle of the inner guide may be proximal to the hub. The handle and the hub may include markings to indicate a rotational orientation of the inner guide relative to the sleeve. In some examples, the inner guide may define three cavities proximate the distal end of the inner guide.

The present disclosure also includes a method of treating a subject, the method comprising positioning a distal portion of a medical instrument proximate a target tissue site of the subject. The medical instrument may comprise a sleeve defining a lumen and an aperture proximate a distal end of the sleeve. An inner guide may be slidable along the lumen of the sleeve and rotatable relative to the sleeve via a handle at a proximal end of the inner guide. The inner guide may define a first cavity and a second cavity each having a shape corresponding to a shape of the aperture of the sleeve. The method may further comprise aligning the first cavity of the inner guide with the aperture of the sleeve; positioning the distal portion of the medical instrument so that a first portion of tissue extends at least partially into the aperture and the first cavity; moving the inner guide in a proximal direction relative to the sleeve to cut the first portion of tissue; rotating the inner guide relative to the sleeve to reposition the first portion of the tissue within the sleeve; aligning the second cavity of the inner guide with the aperture of the sleeve by moving the inner guide in a distal direction; positioning the distal portion of the medical instrument so that a second portion of tissue extends at least partially into the aperture and the second cavity; and moving the inner guide in the proximal direction relative to the sleeve to cut the second portion of tissue. According to some aspects, the distal portion of the medical instrument has a preformed curvature relative to a longitudinal axis of the medical instrument, the aperture being on an inward-facing side of the sleeve relative to the curvature.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 depicts a perspective view of an exemplary medical system according to some aspects of the present disclosure.

FIG. 2 depicts a front plan view of an exemplary medical instrument according to some aspects of the present disclosure.

FIGS. 3A-3C depict exemplary views of the distal portion of the medical instrument of FIG. 2.

FIGS. 3D-3E depict cut-away views of the distal portion of the medical instrument of FIG. 2, without the tip of the inner guide for illustrative purposes.

FIGS. 4A-4B depict cut-away views of another exemplary distal portion of a medical instrument, according to some aspects of the present disclosure.

FIGS. 5A-5H illustrate steps of an exemplary method of obtaining tissue samples using a medical instrument, according to some aspects of the present disclosure.

FIG. 6 depicts an exemplary proximal portion of a medical instrument, according to some aspects of the present disclosure.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Aspects of the present disclosure are now described with reference to exemplary medical instruments, devices, systems, and methods useful for collecting tissue samples, e.g., biopsy samples. The medical instruments herein may facilitate collection of more than one tissue sample without completely withdrawing and reintroducing the instrument for each sample. The medical instrument may include a sleeve and an inner guide. The sleeve may define a lumen, e.g., the sleeve surrounding at least a portion of the inner guide. For example, the inner guide may be moveable and rotatable along the lumen of the sleeve. The inner guide may include a handle to allow a user to control the inner guide, e.g., to translate and/or rotate the inner guide relative to the sleeve. The sleeve may include an aperture proximate a distal end of the sleeve, and the inner guide may include one or more cavities, e.g., at least two cavities, each corresponding to the shape of the aperture. The inner guide and sleeve may allow the user to collect multiple tissue samples, e.g., cutting and storing the tissue samples within the medical instrument.

In an exemplary procedure, a user may introduce the medical instrument into the body of a subject, e.g., through a lumen or other body cavity, and advance the medical instrument until the distal end of the medical instrument is at or proximate a target tissue site. The medical instrument may be maneuvered so that a portion of tissue to be collected extends at least partially into the aperture of the sleeve while the aperture is aligned with a cavity of the inner guide. Subsequently, by manipulating the handle, the user may translate the inner guide relative to the sleeve, e.g., in a proximal direction. The inner guide may engage with and cut the portion of tissue that has entered the aperture and cavity, e.g., via one or more sharp edges of the medical instrument, thereby procuring the initial tissue sample for the biopsy. To obtain another tissue sample, the user can rotate the handle to reposition the previously cut tissue within the sleeve and translate the inner guide distally within the sleeve to align a different cavity of the inner guide with the aperture. The user can again move the inner guide proximally to cut and secure a second tissue sample. Once the tissue samples have been collected within the respective cavities of the inner guide, the user may withdraw the inner guide from the sleeve and/or withdraw the entire medical instrument from the subject in order to analyze the tissue samples.

Reference will now be made in detail to examples of the present disclosure described above and illustrated in the accompanying drawings. Wherever possible, the same reference numbers will be used throughout the drawings to refer to the same or like parts.

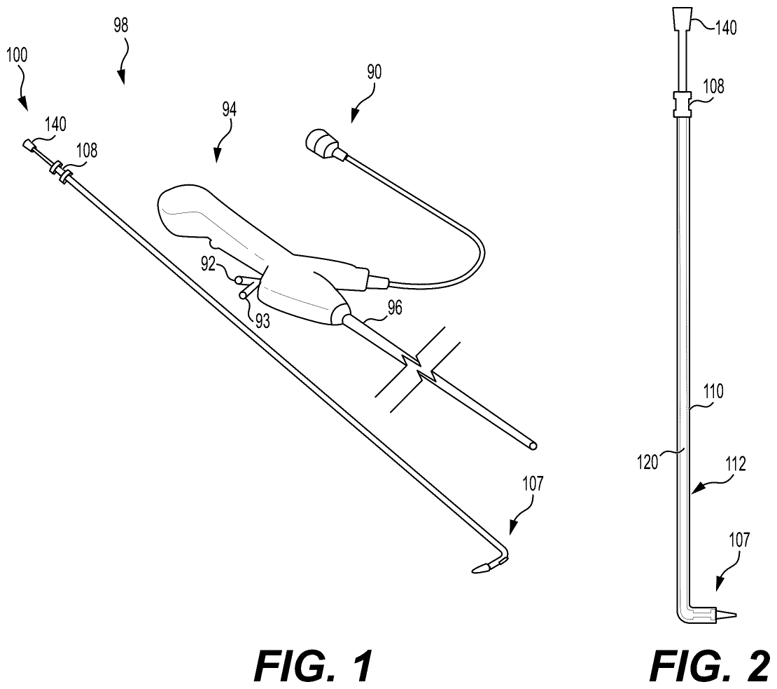

An exemplary medical system 98 according to the present disclosure is illustrated in FIG. 1. The medical system 98 includes an insertion device 90 and a medical instrument 100 insertable into a working channel of the insertion device 90. The insertion device 90 may be any medical device configured to facilitate introduction of the medical instrument 100 into a subject. For example, the insertion device 90 may be an ureteroscope, an endoscope, a hysteroscope, a bronchoscope, a cystoscope, or similar devices defining a working channel. The insertion device 90 may include a handle assembly 94 with one or more ports 92, 93 in communication with one or more working channels defined by a shaft 96 of the insertion device 90 (only a proximal portion of the shaft 96 shown in FIG. 1). The medical instrument 100 may be inserted through one of the ports 92, 93 and advanced through the working channel to exit through a distal opening of the shaft 96. Alternatively, the medical instrument 100 may be used without an insertion device 90, e.g., the medical instrument 100 introduced into the subject directly.

As explained in further detail below, the medical instrument 100 may be flexible. In some aspects, a distal portion 107 of the medical instrument 100 may have a preformed curvature that temporarily straightens when constrained within a working channel, e.g., as the medical instrument 100 passes through the working channel of the insertion device 90. The preformed curvature of the distal portion 107 may form an angle ranging from about 30° to about 120° relative to the longitudinal axis of the medical instrument 100, e.g., an angle ranging from about 30° to about 90°, from about 60° to about 105°, or from about 45° to about 90°. Once the distal portion 107 of the medical instrument 100 exits the distal opening of the insertion device 90, for example, the medical instrument 100 may regain its preformed curvature (e.g., forming a “J” shape in the examples shown in FIG. 1).

FIG. 2 depicts the medical instrument 100, including a sleeve 110, which has a hub 118 at its proximal end. The medical instrument 100 also includes an inner guide 120, which has a handle 140 at its proximal end. The inner guide 120 may be positioned within the sleeve 110, e.g., translatable or slidable along a lumen 112 of the sleeve 110. The sleeve 110 may surround at least a portion of the inner guide 120 along its length. As shown in FIG. 2, the sleeve 110 may be generally tubular in shape, although the present disclosure is not limited to tubes.

Each of the inner guide 120 and the sleeve 110 may comprise suitable biocompatible materials such as, e.g., a polymer, a metal or metal alloy. Exemplary biocompatible materials suitable for the present disclosure include, but are not limited to, Nitinol, stainless steel, and polymers such as polyurethane, silicone, and other plastic materials. The material(s) may provide the medical instrument 100 with sufficient flexibility to navigate through patient anatomy. The flexible materials allow the inner guide 120 and the sleeve 110 to transition between a preformed curvature of the distal portion 107 of the medical instrument 100 and a substantially straight configuration suitable for introduction into a subject. In some examples, the distal portion 107 may have a preformed curvature that is generally “J” shaped. The preformed curvature may be beneficial for navigating anatomical structures, including features of the urinary tract like the bladder, ureter, and/or kidney using a retrograde approach. The preformed curvature of the distal portion 107 may assist a user in accessing and capturing tissue samples at relatively shallow layers, such as urothelial tissue, while minimizing accidental tissue penetration during the procedure.

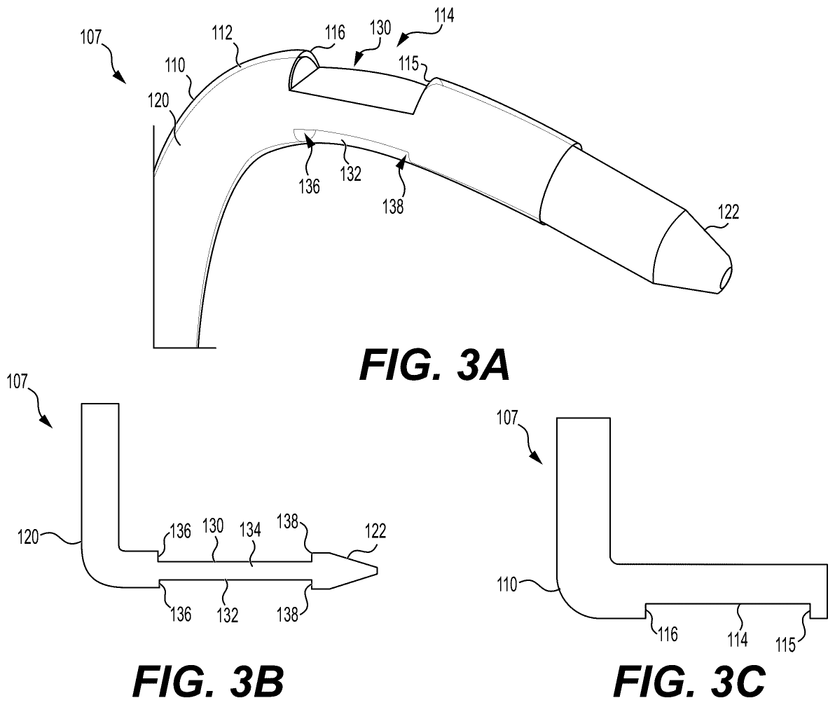

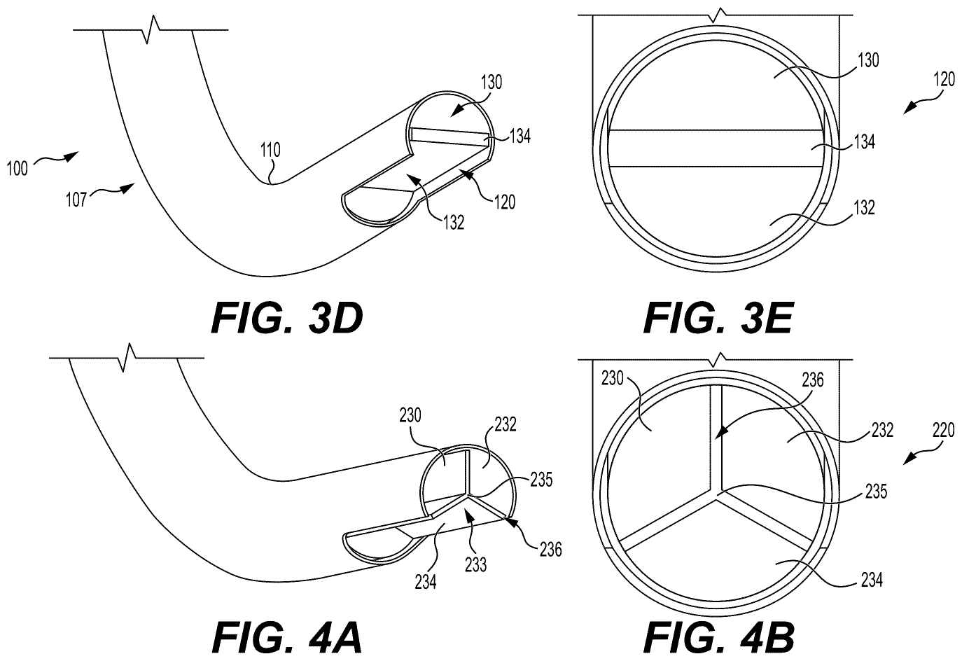

The inner guide 120 includes one or more cavities proximate its distal end. The example shown in FIGS. 2 and 3A-3E shows two cavities 130, 132. The sleeve 110 includes an aperture 114 with a shape corresponding to the shape of each cavity 130, 132. Thus, the aperture 114 may be aligned with one of the cavities 130, 132 at a given time. In some aspects, one or more portions of the cavities 130, 132 of the inner guide 120 and/or the aperture 114 of the sleeve 110 may have a sharp edge or multiple sharp edges to facilitate cutting of tissue, as further described below.

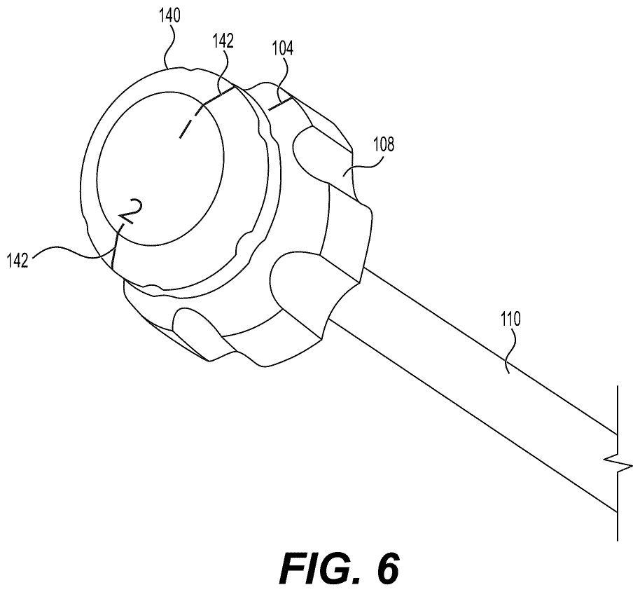

The handle 140 of the inner guide 120 may be used to actuate the inner guide 120, e.g., to translate and/or rotate the inner guide 120 relative to the sleeve 110. The handle 140 may have any suitable shape to be gripped by the hand of a user. For example, the handle 140 may be configured as a knob as shown in FIGS. 1 and 2. The handle 140 of the inner guide 120 may move relative to the hub 108 of the sleeve 110. According to some aspects of the present disclosure, the handle 140 and/or the hub 108 may include one or more markings (e.g., markings 109 of the hub 108 that align with markings 142 of the handle 140; see FIG. 6) to indicate to the user the relative position and/or orientation of the inner guide 120 relative to the sleeve 110. The hub 108 may form the proximal end of the sleeve 110. The hub 108 may be a separate component coupled to the proximal end of the sleeve or may be integral with the material forming the sleeve 110. The hub 108 may serve as a stationary or a central point around which the handle 140 may revolve or move. The handle 140 of the inner guide 120 may move relative to the hub 108 and may include markings 109 that align with markings 142 of the handle 140.

FIG. 3A-3C shows further details of the inner guide 120 and the sleeve 110 forming the distal portion 107 of the medical instrument 100. FIG. 3A shows the inner guide 120 disposed within the sleeve 110. FIG. 3B shows the inner guide 120, and FIG. 3C shows the sleeve 110. The inner guide 120 may have an atraumatic tip 122 distal to the cavities 130, 132. The preformed curvature of the distal portion 107 of the medical instrument 100 (including a distal portion of the sleeve 110 that includes the aperture 114) may be biased in one direction.

The aperture 114 of the sleeve 110 may be a break or an opening in the material of the sleeve 110. While FIGS. 3A-3C show a single aperture 114, in some examples herein, the sleeve 110 may include one aperture 114. For example, the sleeve 110 may include two apertures 114, e.g., a first aperture 114 being proximal to a second aperture 114. The two apertures 114 may have the same shape that aligns with corresponding cavities 130, 132 of the inner guide 120.

In some aspects of the present disclosure, the position of the aperture(s) 114 may be on the outward facing side of the sleeve 110 relative to its curvature. This example is illustrated in FIGS. 3A and 3C. In other aspects, the position of the aperture(s) 114 may be on the inward-facing side of the sleeve 110 relative to its curvature (e.g., the opposite side as that illustrated in FIGS. 3A and 3C). The position of the aperture 114 relative the curvature of the distal portion 107 of the medical instrument 100 may be selected based on the target site of tissue to be collected, e.g., to facilitate navigating the distal portion 107 proximate the target site to collect tissue within the aperture 114.

The aperture 114 may include sharp edges, e.g., along one or more portions of the perimeter of the aperture 114. For example, a distal end 115 of the aperture 114 and/or a proximal end 116 of the aperture 114 may have sharp edges to facilitate cutting of tissue. In some examples, only the proximal end 116 of the aperture 114 includes the sharp edges. In other examples, only the distal end 115 of the aperture 114 includes the sharp edges. The sharp edges may include beveled surfaces or serrated structures that may engage with and cut tissue at the target site.

As mentioned above, the size and shape of the aperture 114 may correspond to the size and shape of the cavities 130, 132 of the inner guide 120. The size and shape of the aperture 114 and cavities 130, 132 may be selected based on characteristics of the tissue target site, e.g., the desired amount of tissue to be collected, the size and/or orientation of the tissue wall, etc. While FIGS. 3A-3C illustrate examples of an aperture 114 and cavities 130, 132 having a generally rectangular cross-sectional shape, the present disclosure is not limited to this example. In other aspects, the aperture 114 and cavities 130, 132 may have other cross-sectional shapes such as, e.g., other polygonal shapes like circular, triangular, hexagonal, etc., or an irregular shape.

The cavities 130, 132 of the inner guide 120 may be configured to engage, receive, and secure tissue samples. Each cavity 130, 132 may include a proximal ledge 136 and a distal ledge 138. In some aspects of the present disclosure, the cavities 130, 132 may include sharp edges to facilitate cutting of tissue. For example, at least a portion of the proximal ledge 136 and/or the distal ledge 138 may have sharp edges. In some examples, only the proximal ledge 136 of each cavity 130, 132, has a sharp edge. In other examples, only the distal ledge 138 of each cavity 130, 132, has a sharp edge. The sharp edges may include beveled surfaces or serrated structures that may prevent slippage during the cutting process, ensuring the tissue is securely held in place while excising.

According to some aspects of the present disclosure, sharp edge(s) of the cavities 130, 132 may cooperate with sharp edge(s) of the aperture 114 to facilitate cutting of tissue samples, e.g., by proximal movement of the inner guide 120 relative to the sleeve 110. For example, sharp edges of the proximal ledge 136 of each cavity 130, 132 may work together with sharp edges of the distal end 115 of the aperture 114 to cut tissue samples as the inner guide 120 moves proximally relative to the sleeve 110.

Each cavity 130, 132 may be separated from adjacent cavities to store tissue samples separately. For example, the inner guide 120 may include a divider 134 between cavities 130, 132. FIGS. 3A-3E illustrate an example of inner guide 120 wherein the cavities 130, 132 are disposed opposite the other, e.g., on either side of the divider 134. For illustrative purposes, FIGS. 3D and 3E depict the distal portion 107 without the atraumatic tip 122 of the inner guide 120. The space defined by each cavity 130, 132 be sized with a width and length large enough for tissue to be cut and stored between the respective cavity 130, 132 and the sleeve 110.

Because the inner guide 120 is slidable within the lumen 112 of the sleeve 110, the tip 122 may be proximal or distal to or the distal end of the sleeve 110. For example, a user may use the handle 140 of the inner guide 120 to move the tip 122 distal to the distal end of the sleeve 110. As mentioned above, the tip may have an atraumatic shape to avoid damage to tissue. For example, the tip 122 may be closed with a generally tapered or curved shape to minimize tissue trauma while navigating the medical instrument 100 through a subject's anatomy.

FIGS. 4A and 4B depict a distal portion 207 of another exemplary medical instrument 200 that includes an inner guide 220 and a sleeve 210. Like FIGS. 3D and 3E, the tip of the inner guide 220 is omitted for illustrative purposes to show cavities of the inner guide 220. The medical instrument 200 may include any of the features of the medical instrument 100 discussed above, except as specified herein. In this example, the inner guide 220 has a plurality of cavities, for example, three cavities 230, 232, 234. The cavities 230, 232, 234 may include any of the features of the cavities 130, 132 described above including one or more sharp edges. The sleeve 210 may radially surround the inner guide 220, similar to the sleeve 110. The three cavities 230, 232, 234 are separated from one another to allow a user to collect and store separate tissue samples. As shown, the cavities 230, 232, and 234 may be positioned about 120° relative to one another although the cavities 230, 232, 234 may be disposed at other intervals. The separation between adjacent cavities 230, 232, 234 may be provided by a divider 233 that forms walls of the cavities 230, 232, 234. For example, the divider 233 may have a “Y” shape with a stem 235 and a plurality of arms 236. The stem 235 may be positioned along a central axis of the inner guide 220.

Referring to the exemplary medical instrument 100 of FIGS. 1-3E, features of the handle 140 and the hub 108 are shown in FIG. 6. As discussed above, the handle 140 may be actuated (e.g., pushed or pulled) to move the inner guide 120 proximally and distally along the lumen 112 of the sleeve 110. The handle 140 may include one or more markings 142 to provide information to a user regarding the orientation of the inner guide 120 relative to the sleeve 110, e.g., to align each cavity 130, 132 with the aperture 114 of the sleeve 110. The hub 108 of the sleeve 110 may also include one or more markings 109 that correspond to the position of the aperture 114. The marking(s) 142 may indicate a first and a second position signified by the number one or two. The respective marking(s) 142 may correspond to one of the cavities 130 or 132. The user may rotate the handle 140, which rotates the inner guide 120 relative to the hub 108 and the sleeve 110. As the user rotates the inner guide 120, marking(s) 142 may align with the marking 109 on the hub 108. For example, the user may rotate the handle 140 and the inner guide 120 until the marking 142 aligns with the marking 109. The user now knows that the cavity 130 or 132 corresponding to the marking 142 (e.g., the number “1”) is aligned with the aperture 114 of the sleeve 110. Further, markings may provide information regarding the longitudinal position of the inner guide 120 relative to the sleeve 110. For example, a portion of the inner guide 120 proximate the handle 140 includes at least one marking to indicate a longitudinal position of the inner guide 120 relative to the sleeve 110.

Optionally, the medical instrument 100 may also include one or more internal stops. For example, an outer surface of the inner guide 120 and/or an inner surface of the sleeve 110 may include a protrusion that limits rotation of the inner guide 120 relative to the sleeve 110. The internal stop(s) may assist a user in aligning a respective cavity 130, 132 with the aperture 114, e.g., to avoid over-rotation and misalignment. The internal stop(s) may comprise materials and/or structures that provide resistance to rotation or otherwise produce tactile feedback to the user when a specific position is reached (e.g., marking 142 “1” or marking 142 “2” of the handle 140 aligns with marking 109 of the hub 108).

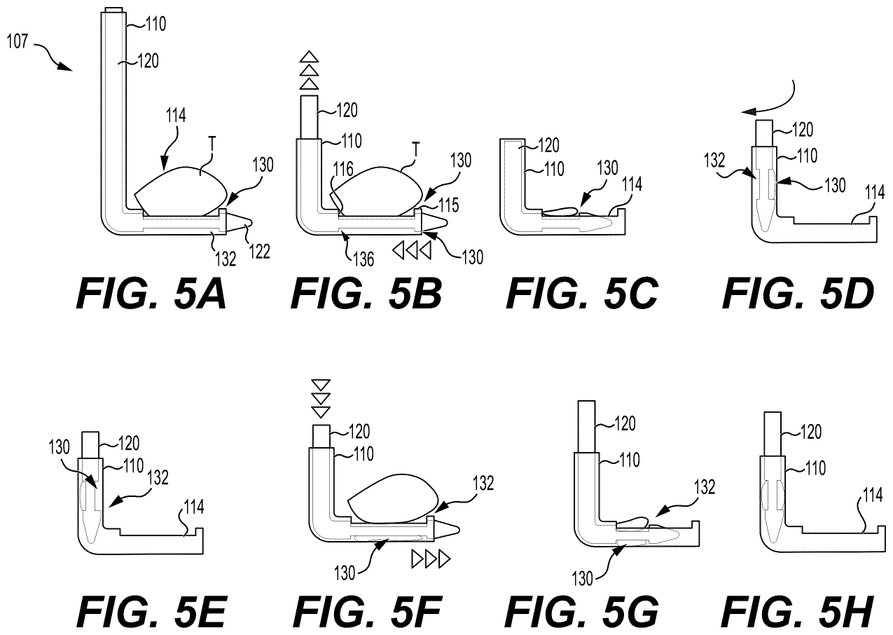

FIGS. 5A-5H illustrate steps of an exemplary method to collect multiple tissue samples using medical instrument 100. The method may be used by a user (e.g., a medical profession) when treating a subject. A user may first introduce the medical instrument 100 into the body of a subject, e.g., through a lumen, organ, or other body cavity, and advance the medical instrument 100 until the distal portion 107 of the medical instrument is at or proximate a target tissue site. In this example, the aperture 114 is on an inward-facing side of the sleeve 110 relative to the curvature, and the curvature is an angle of about 90°. As will be apparent to one of ordinary skill in the art, in other examples, the aperture 114 may be on an outward-facing side of the sleeve 110 relative to the curvature of the sleeve 110 and/or the curvature may be an angle less than 90° (e.g., an angle of about 30°, about 45°, or about 60°) or greater than 90° (e.g., an angle of about 105° or about 120°).

As shown in FIG. 5A, the medical instrument may be maneuvered so that a portion of tissue (“T”) extends at least partially into the aperture 114 of the sleeve 110 while the aperture 114 is aligned with cavity 130 of the inner guide 120. As depicted by the arrows in FIG. 5B, the user may subsequently manipulate the handle 140 to translate the inner guide 120 relative to the sleeve 110, e.g., in a proximal direction. The inner guide 120 and/or the sleeve 110 may engage with and cut the tissue “T” portion that has entered the aperture 114 and the cavity 130, shown in FIG. 3D. Then as shown in FIG. 5D, the inner guide 120 may be further translated in a proximal direction until, for example, the cavity 130 is within the sleeve 110, and the cavity 130 is no longer aligned with the aperture 114. The initial tissue sample may now be stored in the cavity 130.

To obtain another tissue sample, the user may rotate the handle 140 to reposition the inner guide 120 to align the radially position of the cavity 132 with the radial position of the aperture 114. As depicted by the arrows in FIG. 5E and 5F, the user may subsequently manipulate the handle 140 to translate the inner guide 120 distally until the cavity 132 is aligned with the aperture 114. The user can again move the inner guide 120 proximally (FIG. 5G) to cut and secure a second tissue sample. As shown in FIG. 5H, once both tissue samples have been collected within the respective cavities 130, 132 of the inner guide 120, the user may withdraw the inner guide 120 from the sleeve 110. The user may also fully withdraw the medical instrument 100 from the subject in order to analyze the tissue samples.

A similar method may be employed to obtain more than two tissue samples using an inner guide with more than two cavities. For example, the medical instrument 200 may be used in a similar fashion to retrieve a third tissue sample.

The medical instruments herein may facilitate the retrieval of multiple tissue samples without the need to withdraw the instrument between the retrieval of each sample. Further, the medical instruments herein may reduce the duration of procedures to collect multiple tissue samples and/or may reduce the number of times a user may have to insert a medical instrument and find a difficult-to-access tissue site.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical instrument comprising:
a sleeve defining a lumen and an aperture proximate a distal end of the sleeve; and
an inner guide slidable along the lumen of the sleeve and rotatable relative to the sleeve via a handle at a proximal end of the inner guide, a distal end of the inner guide having a closed atraumatic tip;
wherein the inner guide defines at least two cavities proximate the distal end of the inner guide and a divider between each cavity and an adjacent cavity; and
wherein each cavity of the inner guide has a shape corresponding to the aperture of the sleeve, the inner guide being rotatable relative to the sleeve to align one of the at least two cavities with the sleeve to retrieve a tissue sample by proximal or distal movement of the inner guide relative to the sleeve.

2. The medical instrument of claim 1, wherein a distal portion of the medical instrument that includes the inner guide and the sleeve has a preformed curvature relative to a longitudinal axis of the medical instrument.

3. The medical instrument of claim 2, wherein the preformed curvature forms an angle ranging from about 30° to about 90° relative to the longitudinal axis.

4. The medical instrument of claim 1, wherein the inner guide defines two cavities disposed on opposite sides of the inner guide.

5. The medical instrument of claim 1, wherein each cavity of the inner guide includes a proximal ledge and a distal ledge, wherein at least one of the proximal ledge or the distal ledge has a sharp edge configured to cut tissue.

6. The medical instrument of claim 1, wherein at least a portion of the sleeve defining the aperture has a sharp edge configured to cut tissue.

7. The medical instrument of claim 6, wherein the sharp edge is a serrated edge.

8. The medical instrument of claim 1, wherein the inner guide comprises Nitinol, stainless steel, or a polymer.

9. The medical instrument of claim 1, wherein the sleeve comprises a polymer.

10. The medical instrument of claim 1, wherein the handle includes a knob.

11. The medical instrument of claim 1, wherein a proximal end of the sleeve includes a hub, the handle of the inner guide being proximal to the hub.

12. The medical instrument of claim 11, wherein the handle and the hub include markings to indicate a rotational orientation of the inner guide relative to the sleeve.

13. The medical instrument of claim 11, wherein a portion of the inner guide proximate the handle includes at least one marking to indicate a longitudinal position of the inner guide relative to the sleeve.

14. The medical instrument of claim 1, wherein an internal stop between the inner guide and the sleeve limits rotation of the inner guide relative to the sleeve.

15. The medical instrument of claim 1, wherein the inner guide defines three cavities proximate the distal end of the inner guide, the three cavities being disposed at 120° relative to one another.

16. A medical instrument comprising:
a sleeve defining a lumen and an aperture proximate a distal end of the sleeve; and
an inner guide slidable along the lumen of the sleeve and rotatable relative to the sleeve via a handle at a proximal end of the inner guide, the inner guide defining at least two cavities proximate the distal end of the inner guide, each cavity of the at least two cavities being separated from an adjacent cavity and including a sharp edge configured to cut tissue;
wherein each cavity of the inner guide has a shape corresponding to a shape of the aperture of the sleeve, the inner guide being rotatable relative to the sleeve to align one of the at least two cavities with the sleeve to retrieve a tissue sample by proximal or distal movement of the inner guide relative to the sleeve.

17. The medical instrument of claim 16, wherein a proximal end of the sleeve includes a hub, the handle of the inner guide being proximal to the hub, wherein the handle and the hub include markings to indicate a rotational orientation of the inner guide relative to the sleeve.

18. The medical instrument of claim 16, wherein the inner guide defines three cavities proximate the distal end of the inner guide.

19. A method of treating a subject, the method comprising:
positioning a distal portion of a medical instrument proximate a target tissue site of the subject, the medical instrument comprising:
a sleeve defining a lumen and an aperture proximate a distal end of the sleeve; and
an inner guide slidable along the lumen of the sleeve and rotatable relative to the sleeve via a handle at a proximal end of the inner guide, the inner guide defining a first cavity and a second cavity each having a shape corresponding to a shape of the aperture of the sleeve;
aligning the first cavity of the inner guide with the aperture of the sleeve;
positioning the distal portion of the medical instrument so that a first portion of tissue extends at least partially into the aperture and the first cavity;
moving the inner guide in a proximal direction relative to the sleeve to cut the first portion of tissue;
rotating the inner guide relative to the sleeve to reposition the first portion of the tissue within the sleeve;
aligning the second cavity of the inner guide with the aperture of the sleeve by moving the inner guide in a distal direction;
positioning the distal portion of the medical instrument so that a second portion of tissue extends at least partially into the aperture and the second cavity; and
moving the inner guide in the proximal direction relative to the sleeve to cut the second portion of tissue.

20. The method of claim 19, wherein the distal portion of the medical instrument has a preformed curvature relative to a longitudinal axis of the medical instrument, the aperture being on an inward-facing side of the sleeve relative to the curvature.
